(19) United States
(12) Patent Application Publication
(10) Pub. No.: US 2025/0327019 A1
(43) Pub. Date: Oct. 23, 2025

(54) IMMOBILIZED MINING MICROBIAL ACCELERATOR BASED ON BIOSURFACTANT BACTERIA AND PREPARATION METHOD THEREOF

(71) Applicant: Shandong University of Science and Technology, Qingdao (CN)

(72) Inventors: Xiangming HU, Qingdao (CN); Jindi LIU, Qingdao (CN); Yanyun ZHAO, Qingdao (CN); Yue FENG, Qingdao (CN); Xiao LI, Qingdao (CN); Mingyue WU, Qingdao (CN); Yongxiang GUO, Qingdao (CN); Xiaoniu YU, Qingdao (CN); Weimin CHENG, Qingdao (CN); Yu LIU, Qingdao (CN)

(21) Appl. No.: 19/245,153

(22) Filed: Jun. 20, 2025

(30) Foreign Application Priority Data
Apr. 23, 2024 (CN) 202410491804.X

(51) Int. Cl. C12N 1/20; C12N 11/02; E21F 5/06

(52) U.S. Cl. CPC C12N 1/20; C12N 11/02; E21F 5/06

(57) ABSTRACT

The present disclosure relates to an immobilized mining microbial accelerator based on biosurfactant bacteria, including immobilized biosurfactant bacteria, immobilized mineralized bacteria, an activating solution and a cementing agent. The immobilized mining microbial accelerator based on biosurfactant bacteria is prepared by using the following steps: firstly, inoculating biosurfactant bacteria and mineralized bacteria into the activating solution to obtain a biosurfactant bacterium fermentation solution and a mineralized bacterium fermentation solution; and then, selectively adsorbing and enriching the biosurfactant bacteria and the mineralized bacteria through an immobilized material to obtain the immobilized biosurfactant bacteria and the immobilized mineralized bacteria. The hydrophobicity of the immobilized mining microbial accelerator can be improved when the microbial accelerator is applied to coal dust, and further, the improvement of the mineralization capacity and the transformation of the shape of CaCO₃ crystals can be achieved through mutualism between two strains of bacteria.

### CROSS-REFERENCE TO RELATED APPLICATIONS

The present application claims the priority of Chinese patent application entitled “IMMOBILIZED MINING MICROBIAL ACCELERATOR BASED ON BIOSURFACTANT BACTERIA AND PREPARATION METHOD THEREOF” submitted on Apr. 23, 2024, with the application number of 202410491804.X, the entire contents of which are incorporated herein by reference.

### TECHNICAL FIELD

The present disclosure relates to an immobilized mining microbial accelerator based on biosurfactant bacteria and a preparation method thereof, belonging to the technical field of dust prevention and suppression in a coal mine.

### BACKGROUND

Coal dust pollution causes the annual economic loss of up to $1.2 billion in China. Therefore, in order to solve the problem of the coal dust pollution, it is urgent to research and develop a dust suppressant with no secondary pollution, good dust suppression effect and low price. A chemical dust suppressant is formed by combining a variety of chemical substances, which will inevitably adversely affect growth characteristics of microorganisms in an original environment, and is not easy to degrade and easily causes the secondary pollution. The microbial induced carbonate precipitation (MICP) technology is a common phenomenon in nature. It has been reported that by applying the MICP technology to the coal dust, the resulting carbonate precipitates with a gelation effect can bond coal dust particles to form a consolidated layer, and finally the coal dust control is achieved. However, the surface of the coal dust contains many hydrophobic groups (such as aliphatic hydrocarbons and aromatic hydrocarbons), which leads to the problems of poor wettability and difficult penetration when the dust suppressant is applied to the coal dust. At present, a large number of studies have shown that the application of the dust suppressant in the field of the coal dust control requires the addition of a surfactant, the surfactant can improve the wettability of a solution, thereby increasing the contact between a dust suppression material and the coal dust. When a microbial dust suppressant is sprayed, if no surfactant is added, the dust suppressant is difficult to penetrate, and the produced carbonate precipitates can only consolidate the surface of the coal dust with poor consolidation effect, which is easy to cause the reentrainment of the coal dust.

The biosurfactant is a natural surfactive compound synthesized by secretion and metabolism of the microorganisms and has heterogeneous secondary metabolites improving the surface wetting performance of substances. Compared with a chemical surfactant, the biosurfactant has the characteristics of environmental friendliness, easy degradation and the like. Therefore, the penetration, adsorption and retention of the microbial dust suppressant in the coal dust can be enhanced due to the addition of the biosurfactant. How to apply the combination of the biosurfactant bacteria and the mineralization technology to the field of the coal dust control with the microorganisms has become an urgent problem to be solved to promote the development of the microbial dust suppressant in the field of the coal dust control.

### SUMMARY

In view of the problem, the present disclosure provides an immobilized mining microbial accelerator based on biosurfactant bacteria and a preparation method thereof. The immobilized mining microbial accelerator can generate a biosurfactant in a fermentation process to improve the hydrophobicity of coal dust while promoting the mineralized bacteria to develop the mineralization effect by the mutualism of the bacteria to improve the dust suppression effect. In addition, the biosurfactant bacteria negatively charged on surfaces thereof can act as nucleation sites of carbonate precipitates in a microbial induced carbonate precipitation process to accelerate the carbonate precipitation.

In order to achieve the purpose of the present disclosure, the present disclosure provides the following technical solution.

An immobilized mining microbial accelerator based on biosurfactant bacteria includes immobilized biosurfactant bacteria, immobilized mineralized bacteria, an activating solution and a cementing agent, wherein the activating solution and the cementing agent are respectively independently packaged before the use.

It is preferred that a ratio of the total mass of the immobilized biosurfactant bacteria and the immobilized mineralized bacteria to volumes of the activating solution and the cementing agent is (1 g to 3 g) to (65 mL to 75 mL) to (5 mL to 10 mL).

It is preferred that a preparation method of the immobilized biosurfactant bacteria or the immobilized mineralized bacteria includes the following steps:
(1) adjusting the pH of the activating solution with NaOH to be neutral, performing sterilization, then inoculating the activated immobilized biosurfactant bacteria or the activated immobilized mineralized bacteria into the sterilized activating solution, and performing incubation in an incubator to obtain a biosurfactant bacterium fermentation solution and a mineralized bacterium fermentation solution;
(2) respectively sequentially performing operations of centrifugation, removal of a supernate and addition of a sterile saline solution on the biosurfactant bacterium fermentation solution and the mineralized bacterium fermentation solution obtained in the step (1);
(3) after an immobilized material is sterilized, adding a biosurfactant bacterium suspension or a mineralized bacterium suspension, then adding the sterilized activating solution, and performing sealed constant-temperature incubation to obtain an immobilized biosurfactant bacterium solution or an immobilized mineralized bacterium solution; and
(4) filtering the immobilized biosurfactant bacterium solution or the immobilized mineralized bacterium solution via a filter sieve, and lyophilizing the filtered immobilized biosurfactant bacterium solution or the filtered immobilized mineralized bacterium solution by utilizing alyophilizer to obtain the immobilized biosurfactant bacteria or the immobilized mineralized bacteria.

Further, it is preferred that in the step (1), the sterilization is performed at a temperature of 121° C. for 20 min; and the incubation is performed at a temperature of 30° C. and a stirring rotating speed of 150 rpm for 48 h.

Further, it is preferred that in the step (2), the biosurfactant bacterium suspension and the mineralized bacterium suspension have concentrations of 1×10⁷ CFU/mL to 1×10⁸ CFU/mL and 1×10⁸ CFU/mL to 1×10¹⁰ CFU/mL, respectively.

Further, it is preferred that in the step (3), a volume ratio of the biosurfactant bacterium suspension or the mineralized bacterium suspension to the activating solution is 1 to (99 to 105).

Further, it is preferred that in the step (3), the incubation is performed at a temperature of 25° C. and a stirring rotating speed of 150 rpm for 24 h.

Further, it is preferred that a preparation method of the immobilized material includes the following steps:
S1, sequentially performing operations of washing with distilled water, soaking with a NaOH aqueous solution, washing with deionized water and drying to obtain an alkali-treated immobilizer;
S2, adding nanometer titanium dioxide particles and a super-hydrophobic coating material into an activating solution (ethyl alcohol) and uniformly dispersing the nanometer titanium dioxide particles and the super-hydrophobic coating material to obtain a milky white suspension; and
S3, soaking the alkali-treated immobilizer obtained in the step S1 in the suspension obtained in the step S2, and then performing drying to obtain the immobilized material.

Furthermore, it is preferred that in the step S1, the NaOH aqueous solution has a concentration of 1 mol/L to 1.2 mol/L, and the soaking time is 24 h; the number of times of washing is 3 to 5; and a lyophilization method is to perform drying to a constant weight in a vacuum lyophilization oven at a temperature of 65° C.

Furthermore, it is preferred that in the step S1, the immobilizer is at least one of loofah sponge, straw, bagasse and corncob, and has a particle size of 30 mm to 40 mm.

Furthermore, it is preferred that in the step S2, the super-hydrophobic coating material is at least one of paraffin, polytetrafluoroethylene and graphene.

Furthermore, it is preferred that the step S2 is to respectively add 2 g to 5 g of the nanometer titanium dioxide particles and 1 g of the super-hydrophobic coating material into 100 mL of ethyl alcohol and perform ultrasonic dispersion for 2 h to 4 h to obtain the milky white suspension.

Furthermore, it is preferred that in the step S3, the soaking time is 12 h, and the drying is performed in an oven of 60° C. for 24 h.

It is preferred that the biosurfactant bacteria are selected from at least one of Bacillus brevis, Pseudomonas aeruginosa, Bacillus licheniformis or Corynebacterium.

It is preferred that the mineralized bacteria are selected from at least one of jelly-like Bacillus, Bacillus subtilis, Sporosarcina pasteurii, Bacillus amyloliquefaciens or Bacillus sphaericus.

It is preferred that the activating solution contains NH₄Cl, MnSO₄·H₂O and NiCl₂·6H₂O, and further contains a yeast extract or peptone. Further, it is preferred that the activating solution contains the following components in parts by weight: 2000 parts to 3000 parts of peptone, 1000 parts to 2000 parts of NH₄Cl, 100 parts to 150 parts of MnSO₄·H₂O and 200 parts to 300 parts of NiCl₂·6H₂O.

It is preferred that the cementing agent contains a soluble calcium salt and urea.

Further, it is preferred that the soluble calcium salt is at least one of C₂H₂O₄Ca, CaCl₂, C₆H₁₀CaO₆ or C₄H₆CaO₄.

Furthermore, it is preferred that a ratio of molar concentrations of the soluble calcium salt and the urea is 1 to 1.

Furthermore, it is preferred that the soluble calcium salt has a molar concentration of 0.8 mol/L to 1.0 mol/L.

An application of the immobilized mining microbial accelerator based on biosurfactant bacteria for coal dust solidification includes the following steps:
respectively adding immobilized biosurfactant bacteria and immobilized mineralized bacteria into a sterilized activating solution, and performing constant-temperature incubation to obtain a compound microbial fermentation solution; and sequentially spraying the compound microbial fermentation solution and a sterilized cementing agent to coal dust.

It is preferred that a manner of adding the immobilized biosurfactant bacteria and the immobilized mineralized bacteria into the sterilized activating solution is to firstly add 1 part of the immobilized biosurfactant bacteria into a sterilized activating solution and then add 1 part of the immobilized mineralized bacteria after the incubation is performed for 14 h, and continue to perform the incubation for 10 h.

It is preferred that a volume ratio of the compound microbial fermentation solution to the cementing agent is (13 to 15) to (1 to 2).

The present disclosure further provides a production apparatus for the immobilized biosurfactant bacteria or the immobilized mineralized bacteria, which sequentially includes a constant-temperature oscillation incubator, a connection tube, an immobilized bacterium solution storage tank, a filter vibration sieve, a conveyor belt and a lyophilizer according to a direction of a production process. An outlet of the constant-temperature oscillation incubator is connected to an inlet of the connection tube. An outlet of the connection tube is vertically arranged above an opening of the immobilized bacterium solution storage tank. The filter vibration sieve is arranged below an outlet of the immobilized bacterium solution storage tank. One end of the conveyor belt is arranged below the filter vibration sieve and the other end thereof is arranged above an inlet of the lyophilizer.

In the present disclosure, a process of preparing immobilized biosurfactant bacteria or immobilized mineralized bacteria by utilizing the production apparatus includes the following steps:
(1) adding 13 parts to 15 parts of an activating solution and 1 part to 2 parts of a biosurfactant bacterium suspension or a mineralized bacterium suspension into 1 part of an immobilized material, and performing incubation by utilizing a constant-temperature oscillation incubator under incubation conditions of 30° C. and 150 rpm for 48 h to obtain an immobilized biosurfactant bacterium solution or an immobilized mineralized bacterium solution;
(2) conveying the immobilized biosurfactant bacterium solution or the immobilized mineralized bacterium solution into an immobilized bacterium solution storage tank through a connection tube, and making the immobilized biosurfactant bacterium solution or the immobilized mineralized bacterium solution fall into a filter vibration sieve to separate the immobilized biosurfactant bacteria or the immobilized mineralized bacteria from a fermentation solution; and
(3) conveying the immobilized biosurfactant bacteria or the immobilized mineralized bacteria into a lyophilizer through a conveyor belt to lyophilize the immobilized biosurfactant bacteria or the immobilized mineralized bacteria.

Wetting and infiltrating capacities of the immobilized mining microbial accelerator based on biosurfactant bacteria of the present disclosure can be enhanced mainly by using a fact that the biosurfactant bacteria can produce a biosurfactant in a growth process of the bacteria and the biosurfactant can improve the hydrophobicity of the coal dust. The biosurfactant can increase the adhesion effect, causing a large number of microorganisms to adhere to each other to form aggregates. Moreover, the biosurfactant bacteria are negatively charged on surfaces thereof, and can increase nucleation sites to accelerate the carbonate precipitation.

Ammonium ions and carbonate ions can be generated by the decomposition with the addition of a urea reagent, that is, the addition of a decomposition substrate of urease produced by urease-producing bacteria in a liquid environment. A soluble calcium salt can provide calcium ions for the carbonate precipitation, resulting in carbonate precipitates with a gelation effect. That is, the immobilized mining microbial accelerator provided by the present disclosure can effectively provide the basic guarantee for the microbial induced CaCO₃ precipitation, solve the problems of the hydrophobicity and the like faced by the microbial accelerator applied to the coal dust, and further achieve the purpose of environmental protection, no pollution and low cost.

The present disclosure has the following advantages compared with the prior art.
(1) The immobilized mining microbial accelerator of the present disclosure includes the microbial surfactant that is non-toxic to an application environment and microorganisms, thereby enlarging the permeation of the bacterium solutions and a mineralized substrate in a use process, and increasing the thickness of a coal dust solidified layer.
(2) The immobilized material prepared by the present disclosure is hydrophobic, is easy to clean, can be recycled, has a large specific surface area and is beneficial to the uniform distribution of the bacteria.
(3) The urease-producing bacteria can produce the urease in an incubation process, the urease can decompose the urea to produce carbonate ions, and the produced CO₃²⁻ can be bonded to Ca²⁺ in a mineralized solution in a use process to form CaCO₃. In this way, the environmental protection and no pollution are achieved.
(4) The coal dust is solidified by utilizing mineralization and emulsification of the bacteria in the present disclosure without secondary pollution and with low cost and good effect. Accordingly, the immobilized mining microbial accelerator has good application prospect in an open pit coal mine and can be worthy of being popularized.
(5) The immobilized mining microbial accelerator of the present disclosure has the characteristics of convenient construction, environmental protection and no pollution compared with a chemical dust suppressant and a common biological dust suppressant.

### BRIEF DESCRIPTION OF DRAWINGS

FIG. 1 shows growth curves of bacteria in a compound microbial fermentation solution under different inoculation sequences.

FIG. 2 shows amounts of CaCO₃ produced by an immobilized mining microbial accelerator under different inoculation sequences.

FIG. 3 shows electron microscopy views of producing CaCO₃ by an immobilized mining microbial accelerator under different inoculation sequences.

FIG. 4 shows contact angles of an immobilized mining microbial accelerator on coal under different inoculation sequences.

FIG. 5 shows wind erosion resistances of pulverized coal after being treated with an immobilized mining microbial accelerator under different inoculation sequences.

FIG. 6 shows a production apparatus of a material described in an embodiment 1 of the present disclosure, wherein 1, constant-temperature oscillation incubator; 2, connection tube; 3, immobilized bacterium solution storage tank; 4, filter vibration sieve; 5, conveyor belt; and 6, lyophilizer.

### DETAILED DESCRIPTION OF THE EMBODIMENTS

The present disclosure will be further described below in conjunction with specific embodiments, and advantages and characteristics of the present disclosure will become apparent with the description. However, the embodiments are merely exemplary and do not constitute any limitation on the scope of the present disclosure. Those skilled in the art should understand that details and forms of the technical solution of the present disclosure may be modified or replaced without departing from the spirit and scope of the present disclosure, but these modifications and replacements fall within the scope of protection of the present disclosure.

### Embodiment 1

Disclosed are an immobilized mining microbial accelerator based on biosurfactant bacteria and a preparation method thereof.

The preparation method of the immobilized mining microbial accelerator based on biosurfactant bacteria includes the following steps:
I, preparing an immobilized material by the following steps:
S1, washing loofah sponge with distilled water, soaking the washed loofah sponge in a 1 mol/L NaOH aqueous solution for 24 h, washing the loofah sponge 5 times with deionized water to remove the residual NaOH, and then, drying alkali-treated loofah sponge in a vacuum lyophilization oven of 65° C. to constant weight;
S2, respectively adding 2 g of nanometer titanium dioxide particles and 1 g of a paraffin into 100 mL of ethyl alcohol and performing ultrasonic dispersion for 2 h to 4 h to obtain a milky white suspension; and
S3, immersing alkali-treated loofah sponge in the suspension for 12 h, then drying the alkali-treated loofah sponge in an oven of 60° C. for 24 h to obtain the immobilized material;
II, preparing immobilized biosurfactant bacteria and immobilized mineralized bacteria by the following steps:
(1) adjusting the pH of 2000 parts of a yeast extract, 1000 parts of NH₄Cl, 100 parts of MnSO₄·H₂O and 200 parts of NiCl₂·6H₂O to be 7 with 1 mol/L NaOH, performing sterilization in a vertical autoclave of 121° C. for 20 min, respectively inoculating activated Pseudomonas aeruginosa and activated Sporosarcina pasteurii into a sterilized activating solution by utilizing a pipette, and performing incubation in a constant-temperature oscillation incubator 1 under incubation conditions of 150 rpm and 30° C. for 48 h, to obtain a biosurfactant bacterium fermentation solution and a mineralized bacterium fermentation solution respectively;
(2) centrifuging 30 mL of the biosurfactant bacterium fermentation solution and the mineralized bacterium fermentation solution under a condition of 8000 rpm for 5 min to remove a supernatant; adding 5 mL of a sterile saline solution and performing centrifugation under a condition of 8000 rpm for 5 min, removing the supernatant after the washed bacteria are precipitated twice, and adding 5 mL of the sterile saline solution to obtain a homogeneous biosurfactant bacterium suspension and a homogeneous mineralized bacterium suspension;
(3) weighing two parts of 1.0 g, 3.0 g, 5.0 g, 7.0 g and 10.0 g of a washed and dried immobilized material and placing the weighed washed and dried immobilized material into two 100 mL conical flasks, performing sterilization in an autoclave for 20 min, respectively adding 1 mL of the biosurfactant bacterium suspension and the mineralized bacterium suspension to the conical flasks by using a pipette, adding 99 mL of an activating solution into each conical flask and sealing a mouth of the conical flask with a parafilm, and performing incubation under conditions 25° C. and 150 rpm in a constant-temperature oscillation incubator 1 for 24 h to obtain an immobilized biosurfactant bacterium solution and an immobilized mineralized bacterium solution; and
(4) conveying the immobilized biosurfactant bacterium solution and the immobilized mineralized bacterium solution to an immobilized bacterium solution storage tank 3 through a connection tube 2, and filtering the immobilized biosurfactant bacterium solution and the immobilized mineralized bacterium solution by utilizing a filter vibration sieve 4, and conveying the filtered immobilized biosurfactant bacterium solution and the filtered immobilized mineralized bacterium solution to a lyophilizer through a conveyor belt 5 for being lyophilized to obtain the immobilized biosurfactant bacteria and the filtered immobilized mineralized bacteria; and
III, weighing 1 g of the immobilized biosurfactant bacteria, 1 g of the immobilized mineralized bacteria, 75 mL of the activating solution and 5 mL of a cementing agent to obtain an immobilized mining biosurfactant, wherein the activating solution and the cementing agent are respectively independently packaged.

### Experiment 1

The immobilized biosurfactant bacterium solution and the immobilized mineralized bacterium solution obtained in the step II (3) were left to stand. A supernatant was collected and centrifuged under a condition of 2000 rpm for 10 min. A final sample was collected at 1 cm below a surface of the supernatant. The number of microbial cells adsorbed on the surface of an immobilized material was calculated by measuring an OD value of the sample. Results are shown in Table 1.

TABLE 1
Microbial adsorption capacities of immobilized
materials of different masses

| | Mass (g) of immobilized material | | | | |
|---|---|---|---|---|---|
| | 1.0 | 3.0 | 5.0 | 7.0 | 10.0 |
| Adsorption capacity | 25.67 | 40.29 | 39.18 | 32.41 | 21.85 |
| (mg/g) of immobilized | | | | | |
| biosurfactant bacteria | | | | | |
| Adsorption capacity | 17.89 | 26.73 | 28.52 | 23.64 | 15.34 |
| (mg/g) of immobilized | | | | | |
| mineralized bacteria | | | | | |

Table 1 shows that the number of the microbial cells adsorbed by the immobilized material shows a trend of first increasing and then decreasing with the increase of the mass of the immobilized material. The adsorption capacity is first increased and then decreased with the increase of the mass of coal dust, which may be due to a fact that the number of the microbial cells is not sufficient to adapt to the increase of the immobilized material, resulting in the decrease in the overall adsorption capacity. Among them, when the mass of the immobilized material ranges from 3.0 g to 5.0 g, the number of the microbial cells adsorbed by the immobilized material reaches the highest value of 39.18 mg/g to 40.29 mg/g and 26.73 mg/g to 28.52 mg/g, respectively. Therefore, the mass of the immobilized material selected in 100 mL of a bacterium solution (1×10⁸ CFU/mL) ranges from 3.0 g to 5.0 g when the immobilized biosurfactant bacteria and immobilized mineralized bacteria are prepared.

### Experiment 2

The immobilized biosurfactant bacteria and the immobilized mineralized bacteria are the same as in the embodiment 1.

1.0 g, 2.0 g, 3.0 g, 4.0 g, 5.0 g, 6.0 g, 7.0 g and 8.0 g of the immobilized biosurfactant bacteria and the immobilized mineralized bacteria were weighed according to a mass ratio of 1 to 1, and placed in 70 mL of an activating solution after being sterilized. Mouths were sealed with a parafilm. Incubation was performed in a constant-temperature oscillation incubator 1 under conditions of 25° C. and 150 rpm for 48 h. An OD value of a sample was measured. Growth characteristics of the immobilized bacteria of different masses were reflected according to the OD value. Results are shown in Table 2.

TABLE 2
Growth of immobilized bacteria of different masses

| | Mass (g) of immobilized bacteria | | | | | | | |
|---|---|---|---|---|---|---|---|---|
| | 1.0 | 2.0 | 3.0 | 4.0 | 5.0 | 6.0 | 7.0 | 8.0 |
| OD₆₀₀ | 1.287 | 1.293 | 1.307 | 1.286 | 1.279 | 1.274 | 1.268 | 1.251 |

Table 2 shows that the growth of the immobilized bacteria of different masses shows a trend of first increasing and then decreasing with the increase of the mass of the immobilized bacteria, which may be due to a fact that the growth of the bacteria in the activating solution is increased with the increase of the mass of the immobilized bacteria. However, nutrients in the activating solution are limited, which leads to a competitive relationship between the bacteria. The growth of the bacteria is decreased when the initial addition of the immobilized bacteria is too high. Therefore, the total mass of the immobilized biosurfactant bacteria and the immobilized mineralized bacteria ranges from 1.0 g to 3.0 g when the incubation is performed with the immobilized bacteria.

### Experiment 3

1.0 g of immobilized biosurfactant bacteria and/or 1.0 g of immobilized mineralized bacteria were weighed and inoculated into 75 mL of an activating solution after being sterilized, and a group of microbial fermentation solutions was set according to the inoculation time of the bacteria: X, X₁₄P, X₂₄P, P, P₁₄X, P₂₄X and PX, wherein P indicates inoculation of the immobilized biosurfactant bacteria, X indicates inoculation of the immobilized mineralized bacteria, 14 indicates inoculation of one strain of bacteria 14 h after inoculation of another strain of bacteria, and 24 indicates inoculation of one strain of bacteria 24 h after inoculation with another strain of bacteria. The microbial fermentation solutions were placed and incubated under conditions of 150 rpm and 30° C. in a constant-temperature oscillation incubator for 48 h. At set intervals within 48 h, the microbial fermentation solution in a 200 L conical flask was taken from an ultra-clean bench, the absorbance at a wavelength of 600 nm was measured by using a microplate reader, and growth curves of the bacteria are reflected. The measured results are shown in FIG. 1, which shows growth curves of the bacteria in a microbial fermentation solution under different inoculation sequences.

### Experiment 4

75 mL of the microbial fermentation solution prepared in the experiment 3 was added to 5 mL of a mixed solution of CaCl₂ (36 g/L) and urea (20 g/L) after being sterilized by a filter head, and placed and incubated under conditions of 150 rpm and 30° C. in a constant-temperature oscillation incubator for mineralization for 7 days. A compound microbial fermentation solution after being subjected to the mineralization was filtered through filter paper, and dried with a drying oven at a temperature of 100° C. The total mass of the filter paper and precipitates after being dried was recorded as M₁. The filter paper and the precipitates were washed with 0.7 mol/L hydrochloric acid. Precipitates of CaCO₃ were removed, dried, weighed, and recorded as M₂. The weight of the CaCO₃ is recorded as M₁-M₂ (Table 3).

TABLE 3
Amounts of CaCO₃ produced by immobilized mining microbial
accelerator under different inoculation sequences

| Treatment | Amounts (g/L) of precipitates of CaCO₃ | | | | | | |
|---|---|---|---|---|---|---|---|
| manner | X | X₁₄P | X₂₄P | P | P₁₄X | P₂₄X | PX |
| M₁ − M₂ | 4.19 ± 0.93 | 1.07 ± 0.25 | 0.96 ± 0.05 | 0.77 ± 0.07 | 10.40 ± 0. 7 | 2.20 ± 0.22 | 4.31 ± 0.19 |

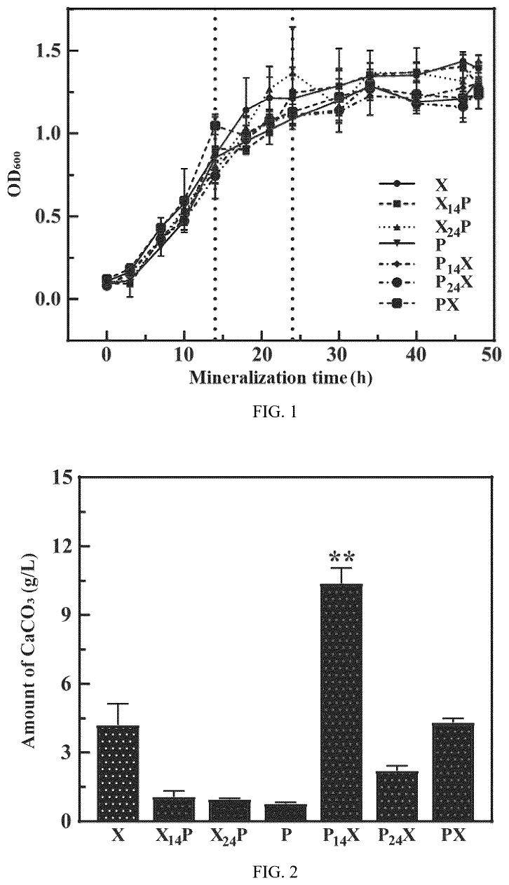

FIG. 2 shows the amounts of the CaCO₃ produced by the immobilized mining microbial accelerator under different inoculation sequences. Results show that after single immobilized bacteria and compound immobilized bacteria are mineralized after 7 days, the amounts of the CaCO₃ produced by the immobilized mineralized bacteria X and the compound immobilized bacteria P₁₄X and PX are high. The amount of the CaCO₃ is 4.19±0.93 g/L after the immobilized mineralized bacteria X are mineralized for 7 days, which is similar to that of the compound immobilized bacteria PX (4.31±0.19 g/L). The amount of the CaCO₃ produced by the compound immobilized bacteria P₁₄X is 10.4±0.70 g/L, which is 148.21% and 141.30% higher than that of the immobilized mineralized bacteria X and the compound immobilized bacteria PX, respectively. The amount of the CaCO₃ produced by the P₂₄X is 2.20±0.22 g/L, which is lower than that of the P₁₄X. These results indicate that the precipitation amount of the CaCO₃ can be increased when the mineralized bacteria are inoculated 14 h after the biosurfactant bacteria are inoculated.

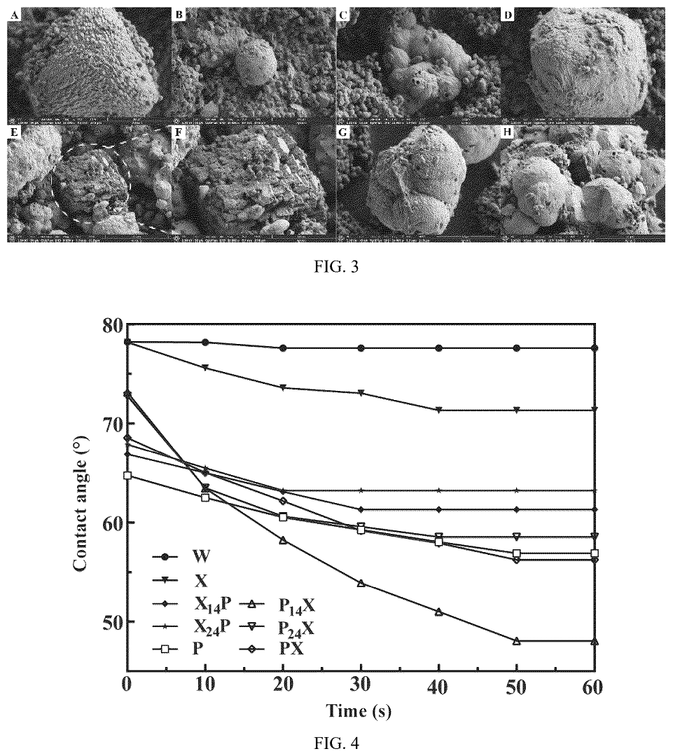

FIG. 3 shows electron microscopy views of CaCO₃ produced by an immobilized mining microbial accelerator under different inoculation sequences, wherein A is X, B is X₁₄P, C is X₂₄P, D is P, E is P₁₄X, F is a local enlarged view of P₁₄X, G is P₂₄X, and H is PX. It can be seen from the figure that a mineralized product produced by single immobilized bacteria is spherical and belongs to vaterite-type CaCO₃. A mineralized product produced by compound immobilized bacteria is vaterite-type CaCO₃ and calcite-type CaCO₃. Among them, the compound immobilized bacteria P₁₄X clearly show the coexistence of the vaterite-type CaCO₃ and the calcite-type CaCO₃, and the vaterite-type CaCO₃ is gradually transformed into the stable calcite-type CaCO₃, indicating that the stability of crystals of the CaCO₃ can be improved when the mineralized bacteria are inoculated 14 h after the biosurfactant bacteria are inoculated.

### Experiment 5

Pulverized coal (1 g, 200 mesh) was pressed into a pulverized coal sample at a pressure of 15 MPa through a tablet press to make a briquette. A contact angle (a seat drop method) test was performed by utilizing an optical contact angle gauge, and effects of biosurfactants produced by bacterium solutions incubated with different inoculation manners (the inoculation manners are the same as in the experiment 3) on the wettability of coal were compared. FIG. 4 shows effects of an immobilized mining microbial accelerator or water (W) on the wettability of coal under different inoculation sequences. Results show that a contact angle of water (W) on the surface of the coal ranges from 78.2° to 77.59°, and a contact angle of an immobilized mineralized bacterium solution (X) on the surface of the coal ranges from 78.160 to 71.33°. The contact angles of the immobilized mineralized bacterium solution (X) and water (W) on the surface of the coal are initially about 78°, but the contact angle of the immobilized mineralized bacterium solution (X) is decreased within 1 min. In addition, the compound bacterium solution P₁₄X has the best wettability for the coal. The tension of the surface of the coal is decreased by 34.27% after 1 min.

### Experiment 6

A certain mass of pulverized coal was weighed and loaded into a graduated cylinder with Φ×h of 10×100 mm, and tamped with a glass rod to make the height of the pulverized coal in the graduated cylinder be 10 cm. Moreover, 5 mL of microbial fermentation solutions under different inoculation manners (the inoculation manners are the same as in the experiment 3) was added for a permeation experiment. The permeation depth within 20 min was measured with a ruler. Results are shown in Table 4.

TABLE 4
Permeation depth of pulverized coal

| | Treatment manner | | | | | | |
|---|---|---|---|---|---|---|---|
| | X | X₁₄P | X₂₄P | P | P₁₄X | P₂₄X | PX |
| Permeation | 1.18 | 3.49 | 3.83 | 2.53 | 5.02 | 4.34 | 4.62 |
| depth (cm) | | | | | | | |

Table 4 shows that the permeation depth of a single immobilized bacterium solution is significantly increased compared with that of a compound immobilized bacterium solution. The permeation depth of the compound immobilized bacterium solution exhibits different permeability with different inoculation sequences. Among them, the permeability of the compound immobilized bacterium solution P₁₄X is significantly higher than that of other compound immobilized bacterium solutions, which indicates that the generation of the immobilized biosurfactant bacteria is promoted when the immobilized mineralized bacteria are inoculated 14 h after the immobilized biosurfactant bacteria are inoculated.

### Experiment 7

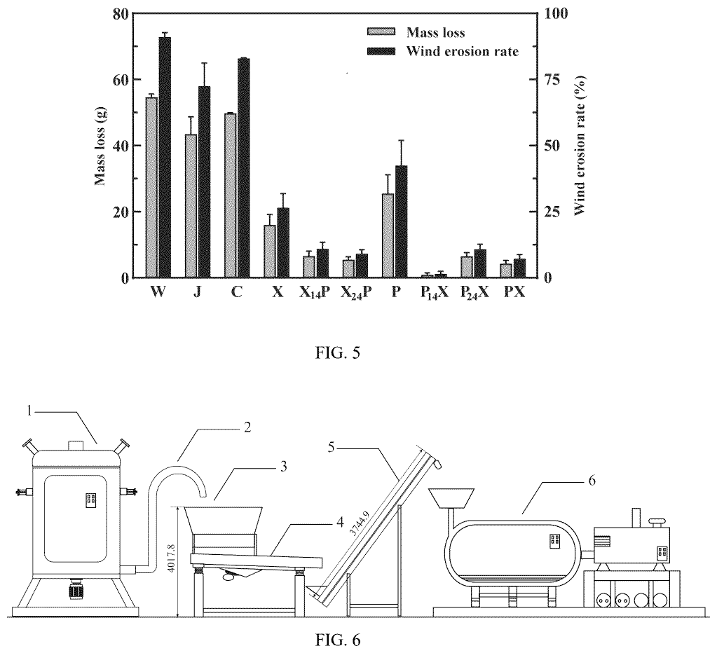

18 mL of microbial fermentation solutions prepared under different inoculation manners (the inoculation manners are the same as in the experiment 3) were weighed and placed in a sprinkling can. 30 g of 120 mesh pulverized coal was weighed with a culture dish, 15 mL of microbial fermentation solutions prepared under different inoculation manners was sprinkled to the pulverized coal in the culture dish, and then 2 mL of a cementing agent, that is, 2 mL of a mixed solution of CaClₕ(36 g/L) and urea (20 g/L) was sprinkled. Finally, the pulverized coal treated with an immobilized mining microbial accelerator was naturally air-dried at room temperature, and the above operations were repeated on the 3^{rd}, 7^{th} and 15^{th} days, respectively. On the 20^{th} day, a wind erosion resistance test was performed on the treated pulverized coal at a wind speed of 10 m/s. Results are shown in FIG. 5. FIG. 5 shows wind erosion resistances of the pulverized coal after being treated with the immobilized mining microbial accelerator under different inoculation manners. As can be seen from the figure that samples treated with water (W), an activating solution (C) and a cementing agent (J) have greater wind erosion mass loss compared to samples treated with the microbial accelerator. In addition, the wind erosion resistance of the compound immobilized bacterium solution is better than that of the single immobilized bacterium solution, which is due to a fact that the mass loss of the samples treated with a compound immobilized bacterium solution is significantly lower than that of samples treated with a single immobilized bacterium solution because of the dual action of microbial mineralization and a biosurfactant. In particular, the microbial accelerator prepared by P₁₄X has good wind erosion resistance (1.55±0.91%), which is reduced by 25.01±4.44% compared with the microbial accelerator prepared by single immobilized mineralized bacteria. Due to different utilization capabilities of microorganisms to substrates, by inoculating and incubating the microorganisms in a certain order, not only can the synergistic metabolic effect between the microorganisms be fully exerted, but also the growth competition and suppression between the microorganisms can be avoided. In this way, higher biomass and yield are achieved.

What is claimed is:

1. An immobilized mining microbial accelerator based on biosurfactant bacteria, comprising immobilized biosurfactant bacteria, immobilized mineralized bacteria, an activating solution and a cementing agent, wherein the activating solution and the cementing agent are respectively independently packaged before the use.
a preparation method of the immobilized biosurfactant bacteria or the immobilized mineralized bacteria comprises the following steps:
(1) adjusting the pH of the activating solution with NaOH to be neutral, performing sterilization, then inoculating the activated immobilized biosurfactant bacteria or the activated immobilized mineralized bacteria into the sterilized activating solution, and performing incubation in an incubator to obtain a biosurfactant bacterium fermentation solution and a mineralized bacterium fermentation solution;
(2) respectively sequentially performing operations of centrifugation, removal of a supernate and addition of a sterile saline solution on the biosurfactant bacterium fermentation solution and the mineralized bacterium fermentation solution obtained in the step (1);
(3) after an immobilized material is sterilized, adding a biosurfactant bacterium suspension or a mineralized bacterium suspension, then adding the sterilized activating solution, and performing sealed constant-temperature incubation to obtain an immobilized biosurfactant bacterium solution or an immobilized mineralized bacterium solution; and
(4) filtering the immobilized biosurfactant bacterium solution or the immobilized mineralized bacterium solution via a filter sieve, and lyophilizing the filtered immobilized biosurfactant bacterium solution or the filtered immobilized mineralized bacterium solution by utilizing alyophilizer to obtain the immobilized biosurfactant bacteria or the immobilized mineralized bacteria; and
a preparation method of the immobilized material comprises the following steps:
S1, sequentially performing operations of washing with distilled water, soaking with a NaOH aqueous solution, washing with deionized water and drying to obtain an alkali-treated immobilizer;
S2, adding nanometer titanium dioxide particles and a super-hydrophobic coating material into an activating solution (ethyl alcohol) and uniformly dispersing the nanometer titanium dioxide particles and the super-hydrophobic coating material to obtain a milky white suspension; and
S3, soaking the alkali-treated immobilizer obtained in the step S1 in the suspension obtained in the step S2, and then performing drying to obtain the immobilized material.

2. The immobilized mining microbial accelerator according to claim 1, wherein a ratio of the total mass of the immobilized biosurfactant bacteria and the immobilized mineralized bacteria to volumes of the activating solution and the cementing agent is (1 g to 3 g) to (65 mL to 75 mL) to (5 mL to 10 mL).

3. The immobilized mining microbial accelerator according to claim 1, wherein in the step (1), the sterilization is performed at a temperature of 121° C. for 20 min; and the incubation is performed at a temperature of 30° C. and a stirring rotating speed of 150 rpm for 48 h.

4. The immobilized mining microbial accelerator according to claim 1, wherein in the step (2), the biosurfactant bacterium suspension and the mineralized bacterium suspension have concentrations of 1×10⁷ CFU/mL to 1×10⁸ CFU/mL and 1×10⁸ CFU/mL to 1×10¹⁰ CFU/mL, respectively.

5. The immobilized mining microbial accelerator according to claim 1, wherein in the step (3), a volume ratio of the biosurfactant bacterium suspension or the mineralized bacterium suspension to the activating solution is 1 to (99 to 105).

6. The immobilized mining microbial accelerator according to claim 1, wherein in the step (3), the incubation is performed at a temperature of 25° C. and a stirring rotating speed of 150 rpm for 24 h.

7. The immobilized mining microbial accelerator according to claim 1, wherein in the step S1, the NaOH aqueous solution has a concentration of 1 mol/L to 1.2 mol/L, and the soaking time is 24 h; the number of times of washing is 3 to 5; and a lyophilization method is to perform drying to a constant weight in a vacuum lyophilization oven at a temperature of 65° C.

8. The immobilized mining microbial accelerator according to claim 1, wherein in the step S1, the immobilizer is at least one of loofah sponge, straw, bagasse and corncob, and has a particle size of 30 mm to 40 mm.

9. The immobilized mining microbial accelerator according to claim 1, wherein in the step S2, the super-hydrophobic coating material is at least one of paraffin, polytetrafluoroethylene and graphene.

10. The immobilized mining microbial accelerator according to claim 1, wherein the step S2 is to respectively add 2 g to 5 g of the nanometer titanium dioxide particles and 1 g of the super-hydrophobic coating material into 100 mL of ethyl alcohol and perform ultrasonic dispersion for 2 h to 4 h to obtain the milky white suspension.

11. The immobilized mining microbial accelerator according to claim 1, wherein in the step S3, the soaking time is 12 h, and the drying is performed in an oven of 60° C. for 24 h.

12. The immobilized mining microbial accelerator according to claim 1, wherein the biosurfactant bacteria are selected from at least one of Bacillus brevis, Pseudomonas aeruginosa, Bacillus licheniformis or Corynebacterium, and have a concentration of 1×10⁷ CFU/mL to 1×10⁸ CFU/mL; and the mineralized bacteria are selected from at least one of jelly-like Bacillus, Bacillus subtilis, Bacillus amyloliquefaciens, Sporosarcina pasteurii or Bacillus sphaericus, and have a concentration of 1×10⁸ CFU/mL to 1×10¹⁰ CFU/mL.

13. The immobilized mining microbial accelerator according to claim 1, wherein the activating solution contains NH₄Cl, MnSO₄·H₂O and NiCl₂·6H₂O, and further contains a yeast extract or peptone.

14. The immobilized mining microbial accelerator according to claim 13, wherein the activating solution contains the following components in parts by weight: 2000 parts to 3000 parts of peptone, 1000 parts to 2000 parts of NH₄Cl, 100 parts to 150 parts of MnSO₄·H₂O and 200 parts to 300 parts of NiCl₂·6H₂O.

15. The immobilized mining microbial accelerator according to claim 1, wherein the cementing agent contains a soluble calcium salt and urea.

16. The immobilized mining microbial accelerator according to claim 15, wherein the soluble calcium salt is at least one of C₂H₂O₄Ca, CaCl₂, C₆H₁₀CaO₆ or C₄H₆CaO₄.

17. The immobilized mining microbial accelerator according to claim 15, wherein the soluble calcium salt has a molar concentration of 0.8 mol/L to 1.0 mol/L.

18. The immobilized mining microbial accelerator according to claim 15, wherein a ratio of molar concentrations of the soluble calcium salt and the urea is 1 to 1.

19. An application of the immobilized mining microbial accelerator according to claim 1 for coal dust solidification, comprising the following steps:
respectively adding immobilized biosurfactant bacteria and immobilized mineralized bacteria into a sterilized activating solution, and performing constant-temperature incubation to obtain a compound microbial fermentation solution; and sequentially spraying the compound microbial fermentation solution and a sterilized cementing agent to coal dust, wherein
a manner of adding the immobilized biosurfactant bacteria and the immobilized mineralized bacteria into the sterilized activating solution is to firstly add 1 part of the immobilized biosurfactant bacteria into a sterilized activating solution and then add 1 part of the immobilized mineralized bacteria after the incubation is performed for 14 h, and continue to perform the incubation for 10 h; and
a volume ratio of the compound microbial fermentation solution to the cementing agent is (13 to 15) to (1 to 2).
